SEQUENCE LISTING (2 sequences)

SEQ ID NO: 1 - protein, 1277 aa, Ciona intestinalis
MSRPDRNQDLPGAYRRSGFSEGRSLPRSSIPPKPSRYTSSYGTSENDFGPDHFNFTDGDTSASGRSRNASTLPSGPSGSSAAFSIRDSHPPPVSDSKRRTISMGANIQPPRSKRSGGTRHRDDEVYDSSDSGIGPRNLTIRPLTRAHANALENNQPPNGPSHNPPPVVRQHRNYREFDHHREQDNHRHTYPPPLPQDDDESFSIAASEKLRDAPPPVYRDDMDFPQMAPLNEDDSCYMGSDDENDWDDYEDFIDDDTPAPETSIPVPKITFKSDNYIYQSLYIFYICSFLAYVFVRIQYTLDAPGLNRIYCIVVAILEIITAPSLILQGLCLWKWVSREPLPDDAYLRQKFHTARIMVPTYKEPLEVVAGTVHEIVHMDLPPNFHVHVYVLDDGRRESLENWVLSKRTKRGVYLHYVARTKLPGVPHHAKAGNINHTLHFVFDDAYAEQECVIIFDADFMPRRNYLLRVLPVFSEKRTRPLALVQTPQFFYNVNSDEDVWDHLNVSFFHRIEPSLDRWSAVNCCGTNFTVRADALKDVGYFPVGCLTEDTLLSLRLCTMGWGVAYHHEVLAIGQSPHEITEIFKQRSRWCKGNLQIFLDEFPLMQSGLSMSQRVFYSSCGFNYFCASISIPFFQLVPAWAIFFGLWPVSKIGLEFAFAFFIYYLLGNFLLLYPPPGFGIKDMWNGELASTNLWFTYFNGVRRIVGTKLLKGKGELTFKTTKKKTDEDEEGSNVGFQKEDVRACYMHFIMFFIMLITIIYAIVKAVTATNVQFYFYYMYMIGMSWALINMTPYLIVVIYCWYRVRIPGLLVACFRNVQLLLRLVCAALILVQAFTTRNLTENFVCPHEYTGQLGASPLAMVKTLDITSNLQVSRNAFWLLGLTDDFYTIQQIKKTACNEDEIPVIVFFMRPSAGLVLDAESSHYMPTDRIASWEEYDVKLENYAKQLADVPSLIVMEPSLLMHTFNSQNSQHDTAYQVQFAQRVQNAANLFSKSWIYVDAGNAMYLQWNVNLNHVINVLKQMPPTIRGFTINVGSFVNSSYNEILASEIHCQTGYHFLIDTSRNGGEFSSRSLDEINSCTYDPPRVRNGSRPTWRDGAATVTLVGQTGLASAAATVPPTETPLPASVKKRWALNAPAAGDPVVEANNPAAGGALPAANAGAAAGGAGGGAAAGNTAAAGGGAAAGAADTSYEYSYDYTAGGATAAVTCVTSEATGLDAYAWIKTPGESDGRMFDAGTYHPCLTDHTTECSETCSQYVTKVDGAFQRSEACQCGTVPEV

SEQ ID NO: 2 - protein, 1274 aa, Oikopleura dioika
MRRSNSSDSFEYTTGPSHRPSNRPEREKTYESIEPNYRDRPPRPSKPPSITTTNRSKTNHNVHSWSRTNNSAPPSNASSFSRPDHARAHDELPSRPSTLNIRSEISRIRAELRQLELEESKSPSEMAELTSLYELQLRKMIHEQVPEDSSIHEFIHPEDTIRPPSRPVKNRAPPPPEQAPSVSTRVSTSNPQNSTKPTPKVRSRQASNKPTSQEPSTQARQRKKIKNEIDDLPPATGKPWIDAAGIERMALEDDGEDWSHFKNFVMDDTPAPEASIPVPKVLYKPTNYIYQFLYIFYLISLLAYIYVRLTFTLDAPGLNRIYCVLVAILEISTCPSLIIQGMTMWKWTSRDEPSLSAFERPKFHTIRVMIPTYNEPLEVVAGTLHEVMHMDVPNGLHLHIYVLDDGKRANLEEYILGRRTKKYVFLHYIARPKLPGVPHHAKAGNINHTLRFIYDRAEAKGEAVVVFDADFMPRRNYLWRVLPVFSEKRDRPIGLVQTPQFFYNVNADEDVWDHLNVSFFHRIEPSLDQWSAVNCCGTNFTVRADALKSVGYFPVGCLTEDTLLSLRLCTEGWAVSYHHEVLAIGQSPHEITEIFKQRSRWCKGNLQIFLDDFPLTQAGLTMSQRIFYSSCGYNYFCAAISIPFFQLVPSWAIFFGLWPVSEIGLEFAFTFLVYYTLGNLLLLYPPPGFQIKDMWNGELASTNLWYTYFNGVRRIVGVKLTQGRGELTFKATKKKAEGDDDDDSGVGFQKEDVRACYMHFIMFFLMLITILYAIIKAIIARSVKFYYYYMYMIGMGWSLINIIPYFIVVIYCWYRTRIPGIMCAILRNVQLLLRGACAILLLIQAFTTRNLTETFICPHPYSATIGTDRLTMIRTLDDVTGKEIERNTFWLLGMKDDFYTIQQIKQSACEDSSVPVVVFYMAPRTGLVLDEQASHYMPPDRLPTWEEYDAKIEEYGEALSEVPIMLILEPSFLMHTFNSENEYHAADYQLSFTQRVDSIIRKFPKAWTYVDAGNALYLQWAVNMDHIVDVLQQMPVGLRGFAMNVGSFVNSTYNMQLASELHCQTGLNYVIDTSRNGGIFSDRSMDEINECTYDPPYISRGAIPGYGPGSKKAINRVDTDLKVDNDYDDYYGDYYYSEYYRKRRSTVPTRHRRQEYEYEEYGYDYYAQYDPAVLECLSNEIEKGHDANSWVKTPGEGDGRLFASGTYHECLLEHTIECDDTCPAYVPKINGEFAREKMCTCDPDELLDYSYDEDVNYENLYFQGHHHHHHHHHH

(19) United States
(12) Patent Application Publication
(10) Pub. No.: US 2018/0094287 A1
(43) Pub. Date: Apr. 5, 2018

(54) COMPOSITION AND METHOD FOR PRODUCING CELLULOSE

(71) Applicant: OKINAWA INSTITUTE OF SCIENCE AND TECHNOLOGY SCHOOL CORPORATION, Okinawa (JP)

(72) Inventors: Keisuke NAKASHIMA, Okinawa (JP); Sakura KIKUCHI, Okinawa (JP); Noriyuki SATOH, Okinawa (JP)

(21) Appl. No.: 15/566,468

(22) PCT Filed: Apr. 20, 2016
(86) PCT No.: PCT/JP2016/002117

§ 371 Date: Oct. 13, 2017

Related U.S. Application Data
(60) Provisional application No. 62/149,667, filed on Apr. 20, 2015.

(51) Int. Cl. C12P 19/04; C12P 19/18; C12N 9/10

(52) U.S. Cl. CPC C12P 19/04; C12N 9/1059; C12Y 204/01012; C12P 19/18

(57) ABSTRACT

A composition that efficiently produces cellulose includes cell extracts derived from a tunicate of ascidian class or yeast expressing tunicate cellulose synthase, at least one divalent cation of calcium ion and magnesium ion, cellobiose and UDP-glucose. The composition has a pH in the range of 6.6-7.2.

### TECHNICAL FIELD

The present invention relates to a composition and method for producing cellulose, specifically, a composition and method for producing cellulose using cell extracts derived from a tunicate of ascidian class or yeast expressing tunicate cellulose synthase.

### BACKGROUND

### Introduction

Cellulose is one of the most abundant materials on Earth and the most common organic polymer. Cellulose is made of linear chains of β (1-4) linked D-glucose. Cellulose has unique crystalline structure which contributes to its usefulness. It is expected that manipulation of the crystalline structure of cellulose will enable us to develop many new biodegradable materials based on cellulose. However, the artificial synthesis of cellulose complete with its crystalline structure has not been successful. Although plant and bacteria produce cellulose, they also have enzymes that synthesize callose or β (1-3) linked D-glucose, and when the enzymes synthesizing cellulose and callose are separated, it is impossible to reassemble the molecular machinery to produce crystalline cellulose. Thus, there is a need to develop a new technology to produce cellulose efficiently, namely, without producing callose.

Tunicates are the only known animals that produce crystalline cellulose without producing callose (Nakashima, K. et al., Dev. Genes Evol., 214: 81-88 (2004), Nakashima, K. et al., Cell. Mol. Life Sci., 68: 1623-1631 (2011)). The inventors of present application succeeded in developing a technology for producing cellulose using cell extracts derived from of a tunicate of ascidian class or yeast expressing tunicate cellulose synthase.

### SUMMARY

One aspect of the present invention provides a composition for producing cellulose, the composition comprising: a cell extract derived from a tunicate of Ascidian or Appendicularian classes, at least one divalent cation of calcium ion and magnesium ion, cellobiose and UDP-glucose, wherein the composition has a pH in the range of 6.6-7.2.

In the composition for producing cellulose, the concentration of the divalent cation may be in the range of 2-8 mM.

In the composition for producing cellulose, the cell extract may be derived from Ciona intestinalis.

In the composition for producing cellulose, the cell extract may be derived from tailbud stage embryos of Ciona intestinalis.

In the composition for producing cellulose, the pH of the composition may be buffered by MOPS.

In the composition for producing cellulose, the pH of the composition may be 6.8.

The composition for producing cellulose may further comprise a stabilizer and/or a protease inhibitor.

In the composition for producing cellulose, the tunicate may express a transgene encoding a protein which is involved in cellulose production.

In the composition for producing cellulose, a cell extract derived from a transformed yeast which may express a transgene encoding a protein which is involved in cellulose production can be used instead of the cell extract derived from a tunicate of Ascidian or Appendicularian classes.

One aspect of the present invention provides a composition for producing cellulose, the composition comprising: a cell extract may be derived from a transformed yeast which may express a transgene encoding a protein which is involved in cellulose production, at least one divalent cation of calcium ion and magnesium ion, cellobiose and UDP-glucose, wherein the composition has a pH in the range of 6.6-7.2.

In the composition for producing cellulose, a cell extract may be derived from a transformed yeast which may express a transgene encoding a cellulose synthase of tunicate.

In the composition for producing cellulose, a cell extract may be derived from a transformed yeast which may contain a protein which is involved in cellulose production.

In the composition for producing cellulose, the protein which is involved in cellulose production may be a recombinant protein derived from the transgene encoding a protein which is involved in cellulose production.

In the composition for producing cellulose, the recombinant protein may be a cellulose synthase of tunicate.

In the composition for producing cellulose, a cell extract may be derived from a transformed yeast which may express a protein of SEQ ID NO: 2.

Another aspect of the invention provides a transformed yeast which may express a transgene encoding a protein which is involved in cellulose production.

Another aspect of the invention provides a transgene encoding a protein which is involved in cellulose production.

Another aspect of the invention provides a method for producing cellulose. The method comprises preparing the composition for producing cellulose, incubating the composition, and purifying the cellulose from the composition.

Another aspect of the invention provides a cellulose which is prepared according to the method for producing cellulose of the present application.

Another aspect of the invention provides a cellulose which exhibits IR spectrum shown in FIG. 2-A.

### BRIEF DESCRIPTION OF DRAWINGS

FIG. 1 illustrates a negatively stained TEM image of cellulose synthesized by the method of the invention (FIG. 1-A) or purified from Ciona intestinalis tissue (FIG. 1-B). The inset in FIG. 1-A shows an electron diffraction pattern of an area of the specimen of 1 μm in diameter.

FIG. 2 illustrates IR spectrum of cellulose which is prepared by the method of the present application (FIG. 2-A) and Avicel (FIG. 2-B).

### DETAILED DESCRIPTION

Various embodiments of the disclosure will be described in detail with reference to drawings, if appropriate. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Unless otherwise indicated, all numbers such as those expressing weight percents of ingredients, dimensions, and values for certain physical properties used in the specification and claims are to be understood as being modified in all instances by the term “about.” It should also be understood that the specific numerical values used in the specification and claims form additional embodiments of the invention. Efforts have been made to ensure the accuracy of the numerical values disclosed in the Examples. Any measured numerical value, however, can inherently contain certain errors resulting from the standard deviation found in its respective measuring technique.

As used herein, the indefinite article “a” or “an” means “at least one,” and should not be limited to “only one” unless explicitly indicated to the contrary. Thus, for example, reference to “a divalent cation” includes embodiments having one, two or more divalent cations, unless the context clearly indicates otherwise.

The term “producing cellulose” as used herein, means the production of linear chains of β (1-4) linked D-glucose that may be amorphous or that may form at least one crystalline structure of cellulose I (or its crystalline phases, triclinic I α and monoclinic I β allomorphs), cellulose II, cellulose III and cellulose IV (Nishiyama, Y., J. Wood Sci., 55: 241-249 (2009), Moon, R. J. et al., Chem. Soc. Rev., 40: 3941-3994 (2011)).

The term “cell extract” means a homogenate which is prepared by disrupting the cell and nuclear membranes of the cells or a microsomal fraction which is separated from soluble components of the homogenate with such a procedure as, but not limited to, an ultracentrifugation. The cell extract of the present invention may be prepared as disclosed herewith. However, alternative means may be employed, for example, a French press instead of a Parr cell disruption bomb. During preparation of the cell extract, a stabilizer such as, but not limited to, 0.5-10 mM of EDTA and/or EGTA or 1-10% of glycerol may be supplemented to the solution containing the homogenate or the microsomal fraction with the activity to synthesize cellulose. During preparation of the cell extract and reaction to produce cellulose, a stabilizer such as 1-10% of glycerol and/or a protease inhibitor such as, but not limited to, PMSF, leupeptin and N-α-p-tosyl-L-Lys chloromethyl ketone may be supplemented in the cell extract and/or reaction mixture. During preparation of the cell extract and reaction to produce cellulose, a mild detergent such as, but limited to, Brij (trade mark) 35, 52, 58, 93, C10, O20 and S100 and Triton (trade mark) X-100 and others may be supplemented. Also during preparation of the cell extract and reaction to produce cellulose, the aqueous solution is buffered by a buffering agent such as, but not limited to Tris, HEPES, MOPS and others, and particularly, MOPS, at the range of pH 6.6-7.2, particularly at 6.8. Unless otherwise noted, the aqueous solution for use in the present invention is based on pure water or ultrapure water, which is known to those skilled in the art.

The term “a tunicate” as used herein refers to an animal of any tunicate species that possess a gene coding cellulose synthetase in its native genome, particularly a species of Ascidian or Appendicularian classes: ascidians such as, but not limited to, Ciona intestinalis, Ciona savignyi, and other species of Ciona genus; Molgula tectiformis, and other species of Molgula genus; Halocynthia roretzi and other species of Halocynthia genus; and appendicularians such as, but not limited to, Oikopleura dioica, Oikopleura longicauda, and other species of Oikopleura genus, and particularly an animal used in the laboratory for genetic studies, such as Ciona intestinalis and Oikopleura dioica.

As the particular species recited above are known to have one or two cellulose synthetase proteins with 65% or more amino acid sequence identity to CiCesA protein of Ciona intestinalis (NCBI Reference Sequence: NP_001041448.1, recited in SEQ ID NO: 1 of the sequence listing attached to the present application), the composition and method for producing cellulose of the present invention works with cell extracts derived from any of the above-mentioned tunicate of Ascidian and Appendicularian classes. According to Nakashima, K. et al. (2004), cellulose synthetase CiCesA is expressed in the epidermal cells. According to Nakamura, M. J. et al. (Dev. Biol. 372: 274-284 (2012)), because epidermal cells constitute about half of entire cells in mid-tailbud stage embryos of Ciona intestinalis, tailbud stage embryos may be a good biological material for preparing the cell extract employed in the composition and method of the present invention. But the composition and method of the present invention will also work with extracts prepared from an entire embryo of any stage, or from any specific epidermal cells of ascidian or appendicularian origin.

As the particular species recited above are known to have one or two cellulose synthase proteins with 65% or more amino acid sequence identity to Od-CesA1 protein of Oikopleura dioica (NCBI Reference Sequence: [AB543594], recited in SEQ ID NO: 2 of the sequence listing attached to the present application).

The term “a transgene encoding a protein that is involved in cellulose production” means a foreign gene or gene construct that is designed to express the foreign gene in a tunicate or a yeast by gene transfer technology such as, but not limited to, microinjection of DNA into a tunicate germline or somatic cell, or a yeast to express the protein permanently or transiently. The foreign gene may encode a protein derived from the same species or derived from a different species, such as a different tunicate species, or a protein derived from plant or bacterial kingdom.

The recipient tunicate may be a wild type tunicate that expresses all the proteins involved in cellulose production, or a tunicate that does not produce cellulose at all or that produces a substantially reduced amount of cellulose compared with a wild type animal, because of induced mutagenesis such as transposon-mediated insertional mutagenesis and oligonucleotide-induced inhibition of protein expression, as reported by Sasakura, Y., et al. (Proc. Natl. Acad. Sci., 42: 15134-15139 (2005)).

A yeast is used as a host, not particularly limited, as long as it belongs to the Ascomycetous yeast. Among them, preferably those are belonging to Saccharomycetaceae, more preferable Saccharomyces.

The term “Expression vector” means a plasmid vector, or it may be an artificial chromosome. An expression vector contains “a transgene encoding a protein that is involved in cellulose production”. When a yeast is used as the host, the form of plasmids is preferred.

The term “a transformed yeast” may be prepared by introducing a polynucleotide or expression vector into a yeast serving as a host. “Introducing” includes not only introduction of a polynucleotide or expression vector but also the gene expression which is introduced into a host cell. Transformation method is not particularly limited, can be adopted a known method. Transformation method includes, for example, calcium phosphate method, electroporation method, lipofection method, DEAE dextran method, a lithium acetate method, transfection methods and microinjection method. A transformed yeast may be selected according to a conventional method such as use of yeast selection marker.

The term “a protein that is involved in cellulose production” means any protein derived from a tunicate, a plant or a bacterium that is involved in cellulose production, such as cellulose synthetase, and a protein involved in the molecular machinery that produces cellulose with at least one of crystalline phases of native cellulose: triclinic I α and monoclinic I β allomorphs.

Chemical purification of tunicate cellulose may be carried out according to any experimental protocols known by those skilled in the art, for example, Nakashima, K. et al (Marine Genomics, 1: 9-14. (2008)). Namely, the whole tunic may be removed from a tunicate specimen. Samples may be treated at high pH, for example, with potassium hydroxide. After neutralization with acid, the samples may be bleached with a chemical such as NaClO₂ buffered at low pH. These steps may be repeated until the samples become white. After washes with distilled water, the samples may be treated with acetic acid/nitric acid aqueous solution (Updegraff, Anal. Biochem. 32: 420-424 (1969)), washed with distilled water, and freeze-dried to yield chemically purified cellulose. Whole embryonic (12 hours post fertilization (hpf)) or larval (18 hpf) specimens may be treated in the same way.

Quantitation of ¹⁴C-labelled cellulose or measurement of its radioactivity may be carried out according to any experimental protocols known by those skilled in the art, for example, Lai-Kee-Him, J. et al. (J. Biol. Chem., 277: 36931-36939. (2002)). Namely, cellulose synthesized in vitro using ¹⁴C-labelled UDP-glucose as a substrate may be recovered by filtration on a glass fiber filter. The glass fiber filter may be washed and dried. The radioactivity may be measured in a liquid scintillation mixture, using a scintillation counter.

TEM and electron diffraction analysis of cellulose specimens such as the cellulose synthesized according to the present invention or purified from a tunicate tissue may be carried out according to any experimental protocols known by those skilled in the art, for example, Lai-Kee-Him, J. et al. (2002). Namely, TEM observations may be achieved using an electron microscope. Specimens may be either negatively stained with uranyl acetate or observed unstained. Low dose electron diffraction patterns may be recorded at liquid nitrogen temperature on unstained specimens, using selected circular areas of 1 μm in diameter. The patterns may be calibrated with a gold standard. A quench-freezing device may be used to prepare cryo-TEM specimens. Drops of detergent extracts, before and after in vitro synthesis, may be deposited on lacy carbon films supported by grids. After blotting the excess of liquid with filter paper, the grids may be immediately plunged into liquid ethane cooled with liquid nitrogen. The excess of ethane may be blotted away with filter paper, and the grids may be mounted on a cryoholder, transferred into the microscope, and observed at such a high magnification as ×11,500, using an under-focus of 1-3 μm.

X-ray diffractometry analysis of cellulose synthesized by the present invention may be carried out according to any experimental protocols known by those skilled in the art, for example, Nakashima, K. et al (2008). Namely, the cellulose may be manually pressed flat. X-ray diffractometry may be performed on a X-ray diffractometer with Cu-Kα radiation (λ=0.15418 nm). An optical slit system may be used. Scanning may be performed at a scattering angle, a scanning step, and a scanning speed that are known to those skilled in the art. Separation of peaks may be performed with software. The diffraction angle may be calibrated with a standard compound such as sodium fluoride. Z values for discriminating crystal allomorphs may be calculated from the d-spacings according to Wada, M. et al. (J. Wood Sci. 47: 124-128 (2001)). Parameters for orientation, R2 and R3, may be calculated from the integrated intensities of peaks according to Koyama, M. et al. (Cellulose 4: 147-160 (1997)).

Attenuated total reflection FTIR may be performed on a FTIR system. The cellulose may be pressed onto the diamond reflector and interferograms may be collected in reflection mode and may be co-added to improve the signal-to noise ratio.

The phrase “having at least 65% sequence identity to a sequence,” as used herein, means that the primer of the present invention has 65% or more, for example, 65, 70, 75, 80, 85, 90, 95, 97, 99% or more, or 100% sequence identity to a particular polypeptide sequence associated with a particular sequence identifier. The sequence identity is determined by aligning the two sequences to be compared as described below, determining the number of identical amino acid residues in the aligned portion, dividing that number by the total number of amino acid residues in the inventive (queried) sequence, and multiplying the result by 100. Polypeptide sequences may be aligned, and the percentage of identical residues in a specified region may be determined against another polypeptide, using a publicly available computer algorithm. Two exemplary algorithms for aligning and identifying the similarity of polynucleotide sequences are the BLASTP and FASTA algorithms. The computer algorithms BLASTP and FASTA are available on the Internet such as The National Center for Biotechnology Information (NCBI) of the U.S. The use of the BLASTP algorithm is described in the publication of Altschul, et al. (Nucleic Acids Res. 25: 3389-3402, 1997). The use of the FASTA algorithm is described in Pearson and Lipman (Proc. Natl. Acad. Sci. USA 85:2444-2448, 1988); and Pearson (Methods in Enzymol. 183: 63-98, 1990).

### Example 1

The present invention is further illustrated by the following non-limiting examples.

Collection of Tunicate Embryos

Ciona intestinalis adults were dissected to obtain eggs and sperm from the gonaduct. After artificial insemination, tunicate embryos were washed with artificial sea water to remove surplus sperm at the two-cell stage, and incubated in the artificial sea water at 18° C. The embryos were collected at late tailbud stage by spinning down

Chemical Purification of Tunicate Cellulose

The whole tunic was surgically removed from adult specimens of Ciona intestinalis. Samples were weighed before and after drying at 60° C. overnight and were then treated with 5% (w/v) potassium hydroxide at 37° C. overnight. After neutralization with 1% (v/v) acetic acid at room temperature for 6 h, the samples were bleached at 80° C. for 2 h with 0.35% NaClO₂ buffered at pH 4.9 with 50 mM sodium acetate buffer. These steps were repeated until the samples became white. After three washes with distilled water, the samples were treated with 73% (v/v) acetic acid/9% (v/v) nitric acid aqueous solution at 95° C. for 30 min (Updegraff, 1969), washed with distilledwater, and freeze-dried to yield chemically purified cellulose. Whole embryonic (12 hours post fertilization (hpf)) or larval (18 hpf) specimens were treated in the same way, each in groups of 1, 10, and 100 individuals.

Preparation of Tunicate Cell Extract

Collected embryos were washed twice with Washing Buffer I (100 mM MOPS (ph7.0), 2 mM EDTA and 2 mM EGTA). The embryos were subjected to two rounds of homogenization with a Parr cell disruption bomb. The supernatant of the homogenate was separated by centrifugation (5,000×g, 10 minutes, 4° C.), followed by filtration with a Miracloth filter (Millipore, Millipore, Merck Ltd.) into an ultracentrifuge tube. After ultracentrifugation (100,000×g, 60 minutes, 4° C.), pellet was collected and suspended in 1 mL of Washing Buffer 11 (100 mM MOPS (pH 7.0), 2 mM EDTA, 2 mM EGTA and 10% (w/v) glycerol). The protein concentration of the tunicate embryo extract was determined with BCA protein assay kit (Pierce, Life Technologies Corporation). The tunicate Embryo Extract was diluted with Washing Buffer II to 6-8 mg/mL and used for the subsequent reactions.

Preparation of Reaction Mixture

Reaction mixture for cellulose synthesis was prepared by mixing 500 microliter of Substrate Buffer (see below) sequentially, first with 50 microliter of 20 mM UDP-glucose (may comprise ¹⁴C-labelled UDP-glucose for radiolabelling experiments), then with 200 microliter of pure water and finally with 250 microliter of the above-mentioned tunicate cell extract. For cellulose synthesis, the reaction mixture was incubated at room temperature for 24 hours.

Optimization of Reaction Condition (1): pH

Washing Buffer II and Substrate Buffer for optimizing pH (50 mM MOPS, 40 mM cellobiose, 4 mM CaCl₂, and 4 mM MgCl₂) were prepared with pH of MOPS adjusted at 6.6, 6.8, 7.0, 7.2, 7.4, and 7.6.

Optimization of Reaction Condition (2): divalent cation concentration

Substrate Buffer for optimizing divalent cation concentration were prepared with 50 mM MOPS (pH 7.0), 40 mM cellobiose, and a combination of Ca Cl₂, Mg Cl₂ and/or Mn Cl₂ at 2, 4 or 8 mM.

Purification of Reaction Product

After completion of the reaction, the reaction mixture was centrifuged (10,000×g, 15 minutes, 20° C.) to remove the supernatant, and the precipitate containing synthesized cellulose was mixed with 1.5 mL of 2% SDS and incubated for one hour at 100° C. The SDS mixture was centrifuged (10,000×g, 15 minutes, 20° C.) to remove the supernatant, and the precipitate was mixed with 1.5 mL of 2% NaOH and incubated for 75 minutes at 100° C. The NaOH mixture was centrifuged (10,000×g, 15 minutes, 20° C.) to remove the supernatant. The precipitate was mixed with 1.5 mL of pure water by inverting the tube a few times. The step of washing the synthesized cellulose with water, comprising centrifuging (10,000×g, 15 minutes, 20° C.) to remove the supernatant and mixing the precipitate containing the synthesized cellulose with a fresh pure water, was repeated five more times. After the repeated steps of washing with water, the synthesized cellulose was subjected to the analyses described below.

Quantitation of ¹⁴C-Labelled Cellulose

Cellulose synthesized with the reaction mixture comprising ¹⁴C-labelled UDP-glucose was recovered by filtration on a glass fiber filter. The glass fiber filter was washed and dried. The radioactivity was measured in a liquid scintillation mixture, using a scintillation counter.

TEM and Electron Diffraction Analysis

TEM observations and electron diffraction analysis may be achieved using an electron microscope for imaging and 200 kV for electron diffraction. Cellulose specimens such as the cellulose synthesized according to the present invention or purified from Ciona intestinalis tissue were either negatively stained with uranyl acetate or observed unstained. Low dose electron diffraction patterns were recorded at liquid nitrogen temperature on unstained specimens, using selected circular areas of 1 μm in diameter.

Fourier Transform Infrared Spectroscopic Analysis

Attenuated total reflection FTIR was performed on a Spectrum One FTIR system (Perkin Elmer). The cellulose synthesized according to the present invention or purified from Ciona intestinalis tissue were pressed onto the diamond reflector at a force gauge of 50, and interferograms (4000-400 cm⁻¹) were collected in reflection mode with 2 cm⁻¹ resolution and were co-added to improve the signal-to noise ratio.

Results

Table 1 below summarizes the results of experiments for optimizing pH of the reaction mixture.

TABLE 1

| pH | activity (dpm) |
|---|---|
| 6.6 | 30413 [78.6] |
| 6.8 | 38714 [100.0] |
| 7.0 | 31764 [82.0] |
| 7.2 | 29642 [76.6] |
| 7.4 | 22495 [58.1] |
| 7.6 | 15526 [40.1] |

Table 1 indicates the measured value for the radioactivity incorporated in the cellulose synthesized at the indicated pH and the percentage value obtained by dividing the measured value at each pH by the measured value for the most radioactive pH, pH 6.8. Table 1 indicates that high synthetic activity was observed over the range from pH 6.6 to pH 7.2, with the highest synthetic activity at pH 6.8.

Table 2 below shows the results of experiments for optimizing divalent cation concentrations of the reaction mixture.

TABLE 2

| conc. (mM) | | | |
|---|---|---|---|
| Ca²⁺ | Mg²⁺ | Mn²⁺ | activity (dpm) |
| 0 | 0 | 0 | 8917 [32.9] |
| 2 | 0 | 0 | 21989 [81.2] |
| 4 | 0 | 0 | 25595 [94.5] |
| 8 | 0 | 0 | 25045 [92.4] |
| 0 | 2 | 0 | 18165 [67.0] |
| 0 | 4 | 0 | 23012 [84.9] |
| 0 | 8 | 0 | 26149 [96.5] |
| 0 | 0 | 2 | 18171 [67.1] |
| 0 | 0 | 4 | 14369 [53.0] |
| 0 | 0 | 8 | 11887 [43.9] |
| 4 | 4 | 0 | 27096 [100.0] |
| 4 | 0 | 4 | 12877 [47.5] |
| 0 | 4 | 4 | 16292 [60.1] |
| 4 | 4 | 4 | 14720 [54.3] |

Table 2 indicates the measured value for the radioactivity incorporated in the cellulose synthesized in each divalent cation concentration and the percentage value obtained by dividing the measured value at each concentration by the measured value for the most radioactive concentration, 4 mM Ca²⁺, 4 mM Mg²⁺ and 0 mM Mn²⁺. Table 2 demonstrates that high synthetic activity was observed at the conditions 2-8 mM of either Ca²⁺ or Mg²⁺, with the highest synthetic activity observed at concentrations of 4 mM Ca²⁺, 4 mM Mg²⁺ and 0 mM Mn²⁺. Mn²⁺ promoted cellulose synthesis in the absence of Ca²⁺ and Mg²⁺. The effect of Mn²⁺ is notable at 2 mM, but less prominent at 4 mM or higher concentration. Mn²⁺ has an inhibitory effect on cellulose synthesis in the presence of Ca²⁺ and/or Mg²⁺.

From these results, it is concluded that a favorable condition for cellulose synthesis in a cell-free system extracted from Ciona intestinalis embryo is pH 6.8, 4 mM of Ca²⁺ and 4 mM of Mg²⁺, with no Mn²⁺.

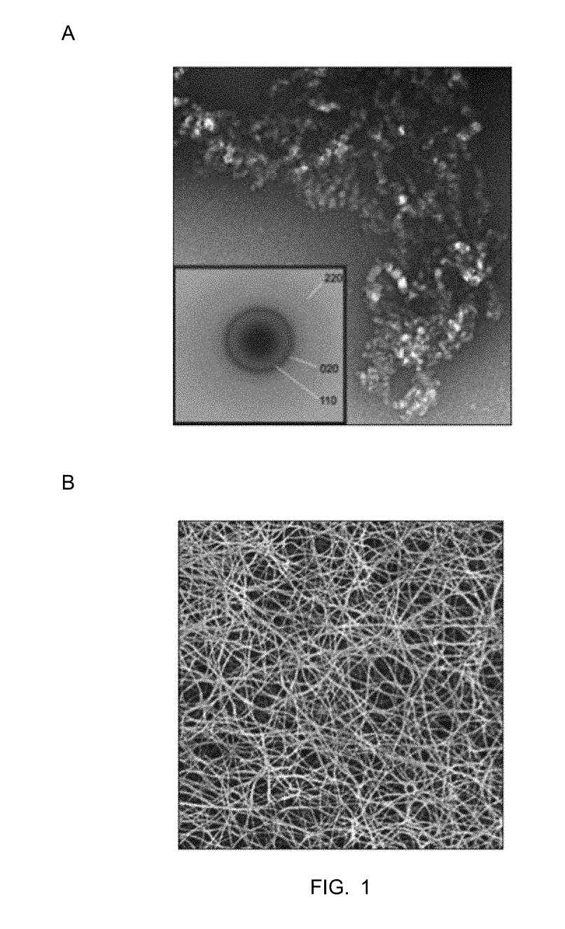

FIG. 1 illustrates a negatively stained TEM image of synthetic cellulose (FIG. 1-A) or purified from Ciona intestinalis tissue (FIG. 1-B). The inset in FIG. 1-A shows an electron diffraction pattern of an area of the synthesized cellulose of 1 μm in diameter. The d-spacing values for lattice planes (110), (020), and (220) were 0.448 nm (n=11), 0.408 nm (n=11) and 0.222 nm (n=3), respectively. These results suggested that the cellulose synthesized in the above example was in Cellulose II structure (anti-parallel chain), and not Cellulose I (all parallel chain), the structure of native cellulose.

### Example 2

1. Construction of Expression Vector

The full-length coding region of Od-CesA1 (SEQ ID NO: 2 of the sequence listing attached to the present application), a cellulose synthetase derived from Oikopleura dioica, was amplified by RT-PCR from a cDNA template using the primer pair OdF and OdR. The amplified fragments were cloned into the pGAPZα expression vector (Life Technologies) by replacing the full-length alpha factor for secretory expression using In-Fusion cloning. The plasmid obtained was named Od1pGZ.

2. Expression of Recombinant Protein Using Yeast Pichia pastoris as Host

Od1pGZ was treated with the AvrII restriction enzyme and was used to transform the yeast Pichia pastoris by the lithium acetate method. The transformed Pichia pastoris was cultured in 5 mL of YPD medium (1% yeast extract, 2% peptone, 2% glucose) containing Zeocin (100 μg/ml) at 30° C. at 200 rpm for 24 hours (preculture), which was subsequently inoculated into 300 mL of YPD medium and cultured at 30° C. at 200 rpm for 24 hours (main culture).

3. Test-Tube Synthesis of Cellulose Using Recombinant Protein

The yeast cells were recovered by centrifugation (4° C., 1,000×g, 3 minutes), suspended in a medium for cell disruption (50 mM sodium phosphate, pH7.4, 5% glycerol, 1 mM EDTA) and then disrupted by subjecting them to three rounds of treatment at 30,000 psi using a LV1 Microfluidizer (Microfluidics). The homogenate was centrifuged (4° C., 1,000×g, 3 minutes) and the recovered supernatant was subjected to ultracentrifugation (4° C., 100,000×g, 60 minutes) to obtain the microsomal fraction as a precipitated pellet. The microsomal fraction was suspended in a solution (75 mM Mops (pH 7.0), 2.5% glycerol, 20 mM cellobiose, 1 mM UDP-glucose, 8 mM MgCl2, 0.5 mM EDTA, 0.5 mM EGTA) and allowed to react at 24° C. for 12 hours.

4. Recovery and Analysis of Synthesized Product

SDS was added to the reaction mixture to a final concentration of 2%, and was subjected to delipidation at 50° C. for 24 hours. The reaction mixture was subjected to centrifugation (28° C., 20,000×g, 20 minutes) and the insoluble fraction was recovered as a precipitate. The precipitate was subjected to protease treatment (Proteinase K at a concentration of 20 μg/1 mL, Phosphate Buffered Saline, 1% SDS, 50° C., 48 hours) and then centrifuged again to recover the precipitate (28° C., 16,000×g, 20 minutes). The precipitate was subjected to alkaline treatment (2% potassium hydroxide, 24° C., 24 hours) and subsequently centrifuged to recover the precipitate (30° C., 16,000×g, 20 minutes). The precipitate obtained was suspended in a mixed solution of acetic acid (72%) and nitric acid (12%) and treated at 100° C. for 30 minutes, followed by dilution with an equal amount of water and then centrifugation (15° C., 16,000×g, 20 minutes) to recover the precipitate. The precipitate recovered was washed several times with water, and then suspended in water to obtain the synthesized product.

5. Analysis Data of Synthesized Cellulose

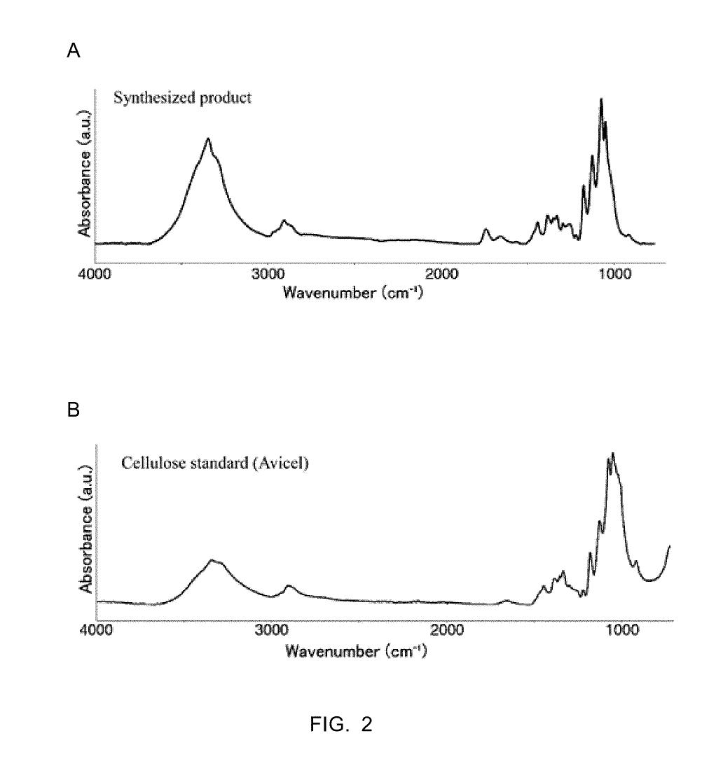

The synthesized product (5 μg) was applied onto a barium fluoride window, and, after drying, subjected to micro FT-IR spectroscopy using a Spotlight 200 (PerkinElmer). FIG. 2 shows the spectra obtained for the synthesized product and a cellulose sample (measured area: 100 μm×100 μm).

It will be apparent to those skilled in the art that various modifications and alterations can be made to the present invention without departing from the scope and spirit of the invention. Thus, it is intended that the present invention cover the modifications and variations of this invention provided they come within the scope of the appended claims and their equivalents.

1. A composition for producing cellulose, comprising:
a cell extract derived from a tunicate of Ascidian or Appendicularian classes, at least one divalent cation of calcium ion and magnesium ion, cellobiose and UDP-glucose, wherein the composition has a pH in the range of 6.6-7.2.

2. The composition of claim 1, wherein the cell extract is derived from Ciona intestinalis.

3. The composition of claim 1, wherein the cell extract is derived from tailbud stage embryos of Ciona intestinalis.

4. A composition for producing cellulose, the composition comprising:
a cell extract may be derived from a transformed yeast which may express a transgene encoding a protein which is involved in cellulose production, at least one divalent cation of calcium ion and magnesium ion, cellobiose and UDP-glucose, wherein the composition has a pH in the range of 6.6-7.2.

5. The composition of claim 4, wherein a cell extract may be derived from a transformed yeast which may express a protein of SEQ ID NO: 2.

6. The composition of claim 1, wherein the concentration of the divalent cation is in the range of 2-8 mM.

7. The composition of claim 1, wherein the pH of the composition is buffered by MOPS.

8. The composition of claim 1, wherein the pH of the composition is 6.8.

9. The composition of claim 1, wherein the composition further comprises a stabilizer and/or a protease inhibitor.

10. The composition of claim 1, wherein the tunicate expresses a transgene encoding a protein that is involved in cellulose production.

11. A method for producing cellulose, comprising:
preparing the composition according to claim 1, incubating the composition, and purifying the cellulose from the composition.

12. A cellulose which is prepared by the method according to claim 11.

13. A cellulose which exhibits IR spectrum:

[symbol]

14. A method for producing cellulose, comprising:
preparing the composition according to claim 4, incubating the composition, and purifying the cellulose from the composition.

15. A cellulose which is prepared by the method according to claim 14.
